(19) United States
(12) Patent Application Publication
(10) Pub. No.: US 2011/0288544 A1
(43) Pub. Date: Nov. 24, 2011

(54) MEDICAL DEVICE FOR TISSUE ABLATION

(75) Inventors: Vitali Verin, Chene-Bourg (CH); Lionel Flaction, Lausanne (CH)

(73) Assignee: MAESTROHEART SA, Chene-Bourg (CH)

(21) Appl. No.: 13/000,475

(22) PCT Filed: Jul. 17, 2009
(86) PCT No.: PCT/IB2009/053123

§ 371 Date: Mar. 15, 2011

Related U.S. Application Data
(60) Provisional application No. 61/081,397, filed on Jul. 17, 2008.
(60) Provisional application No. 61/081,401, filed on Jul. 17, 2008.

(51) Int. Cl. A61B 18/14

(52) U.S. Cl. 606/41

(57) ABSTRACT

A medical device for ablating tissues within a heart chamber comprising a first guiding member intended to be introduced in the hollow structure surrounding the left atrium of the patient and a second ablating member comprising an ablation electrode mounted at the distal end or tip of catheter. Both, the head of the guiding member and the tip of the ablating member are magnetised and can enter into magnetic coupling when their distal ends are brought in close contact. Once the magnetic coupling is achieved, the tip of the first member is guided by moving the guiding member. Preferably, the guiding member includes sensors enabling to monitor physiological parameters during the intervention.

### TECHNICAL FIELD

The present invention relates to an improved medical device or apparatus for ablating cardiac tissues along continuous lines in the heart chambers. A further object of the invention relates to a method for positioning and guiding an ablation catheter during ablation procedure. More particularly the device and method of the present invention are intended to perform ablation lines on the wall of the left atrium in order to treat and prevent the occurrences of atrial fibrillation. The medical device comprises to that extent a first elongated member having a distal end comprising an ablation electrode and a second elongated member allowing precise control of the ablation electrode.

### BACKGROUND ART

Abnormal heart rhythms are generally referred to as cardiac arrhythmias and with an abnormally rapid rhythm called tachycardia. Atrial fibrillation is an abnormal rhythm of the heart caused by abnormal electrical discharges within the two upper chambers of the heart called atria. Atrial fibrillation reduces the ability of the atria to pump blood into the lower chambers of the heart (the ventricles) and usually causes the heart to beat too rapidly and may induce complications that include heart failure and stroke.

While medication has been used to prevent recurrence of atrial fibrillations, they are not always effective and may induce undesirable or intolerable side effects. Furthermore they do not cure the underlying causes. Implantable devices have also been used but they only correct the arrhythmia after it occurs and do not help to prevent it.

Surgical and invasive catheterisation approaches in contrast are promising and give very good results as they cure the problem by ablating the portion of the heart tissue that causes electrical trouble inducing fibrillation.

Before performing ablation of some portion of the inner wall of atria, a cardiac mapping is firstly executed in order to locate aberrant electrical pathways within the heart as well as to detect other mechanical aspects of cardiac activity. Various methods and devices have been disclosed and are commonly used to establish precise mapping of the heart and will not be further described in the present application. Once this mapping is done, the clinician will refer to this heart mapping, which indicates him the points and lines along, which ablation is to be performed.

One commonly used technique for performing ablation is known as radiofrequency catheter ablation. This technique uses an ablation electrode mounted at the distal end of a catheter that is introduced by natural passageways in the target heart chamber and then manipulated by a physician (electrophysiologist, surgeon, etc) thanks to a handle at the proximal end of the catheter acting on a steering mechanism. This allows displacement of the distal end of the catheter so as to have the ablation electrode lying at the exact position determined by the heart mapping technique or/and fluoroscopy. Once the ablation electrode is in contact with the pre-determined area, RF energy is applied to ablate the cardiac tissue. By successfully causing a lesion on the pre-determined portion of the cardiac tissues, the abnormal electrical patterns responsible for the atrial fibrillation are eliminated.

However, this technique presents several difficulties. The currently used techniques of manual catheter ablation as well as robotic ablation systems in development do not allow precise controlled movements of the ablation electrode tip along the internal atrial wall surface. The ablation electrode located at the distal end of the catheter tends to slip and jump from one point to another instead of following a straight line. The absence of real time visualisation of the atrial wall during the intervention hampers the generation of precise continuous ablation lines. The gaps between ablation points are commonly leading to a lack of treatment efficacy and may induce development of atrial flutter.

Another known problem relates to the determination of the correct level of energy to deliver to the ablation tip so as to precisely control the ablation lesion depth. When the catheter distal end is not correctly positioned or when the ablation electrode is not perpendicular to the cardiac tissue, energy applied may be either too low, in that case the lesion is ineffective, or too high which may lead in rare cases to atrial wall perforation, oesophageal burns and atrial-oesophageal fistula formation. This complication, although rare, is extremely devastating and fatal in more than half of the reported cases.

The use of a temperature sensor at the tip of the catheter in the vicinity of the ablating electrode does not help to solve this problem as it does not provide an accurate measure of the tissue temperature because the measure is mostly influenced by the heating of the ablation electrode and its cooling by the irrigation liquid when RF energy is applied.

An ablation device has been disclosed in PCT application WO 2008/010039 and this document is incorporated by reference in its entirety in the present application for the disclosure of such a device. This device includes a medical device for ablating tissues within a heart chamber comprising a first guiding member intended to be introduced in the hollow structures surrounding left atrium (such as oesophagus, pulmonary artery, coronary sinus, aorta, right atrium, pericardial cavity etc) of the patient and a second ablating member comprising an ablation electrode mounted at the distal end or tip of catheter. Both, the head of the guiding member and the tip of the ablating member are magnetised and can enter into magnetic coupling when their distal ends are brought in close contact. Once the magnetic coupling is achieved, the tip of the first member is guided by moving the guiding member. Preferably, the guiding member includes sensors enabling to monitor physiological parameters mostly related to the tissue status during the intervention.

### SUMMARY OF THE INVENTION

An aim of the present invention is to improve the known tissue ablation devices.

More precisely, an aim of the present invention is to improve the ablation device known from the prior art, in particular from WO 2008/010039 as incorporated in the present application.

Another aim of the present invention is to provide a medical device or apparatus and a method that allows the precise control of the positioning and of the movements of the ablation electrode during the intervention and the effective monitoring of the adequate physiological tissue related parameters in order to prevent or even eliminate the occurrence of the above-mentioned dreadful complications.

Another aim of the present invention is to provide a system in which the guiding of one of the used members is made easy and practical.

Another aim of the present invention is to improve the positioning of the devices during use and medical intervention.

A device according to the invention is defined in the independent claims. Other characteristics of the medical apparatus and of the method object of the present invention are recited in the dependant claims.

The details of one or more embodiments of the invention are set forth in the accompanying drawings and in the description below. Other features, objects and advantages of the invention will be apparent from the following detailed description and drawings in which:

### BRIEF DESCRIPTION OF THE DRAWINGS

FIGS. 1 and 2 illustrate the principles of the invention;

FIG. 3 illustrates an embodiment of the invention;

FIGS. 4-6 illustrate variants of the present invention;

FIGS. 7, 7A, 8, 8A illustrate an embodiment of the present invention;

FIGS. 9 and 9A illustrate an embodiment of the present invention;

FIG. 10 illustrates another embodiment of the device according to the invention;

FIG. 11 illustrates another embodiment of the device according to the invention;

FIGS. 12 and 12A illustrate a variant of the invention;

FIGS. 13 and 13A illustrate another variant of the invention;

FIGS. 14 and 14A illustrate a further variant of the invention;

FIGS. 15 and 15A illustrate a further variant of the invention;

FIGS. 16 and 16A illustrate a further variant of the invention;

FIGS. 17 and 17A illustrate a further variant of the invention;

FIG. 18 illustrates another embodiment of the invention;

FIG. 19 illustrates a specific embodiment of the invention;

FIG. 20 illustrates a first example of use of the device according to the invention;

FIG. 21 illustrates a another example of use of the device according to the invention

FIG. 22 illustrates a further example of use of the device according to the invention;

FIG. 23 illustrate another embodiment of the device according to the invention and

FIG. 24 illustrates another embodiment of the device according to the invention.

### DETAILED DESCRIPTION

For the basic description of the device according to the invention, reference is made to WO 2008/010039 mentioned above in the present specification and incorporated in its entirety in the present application.

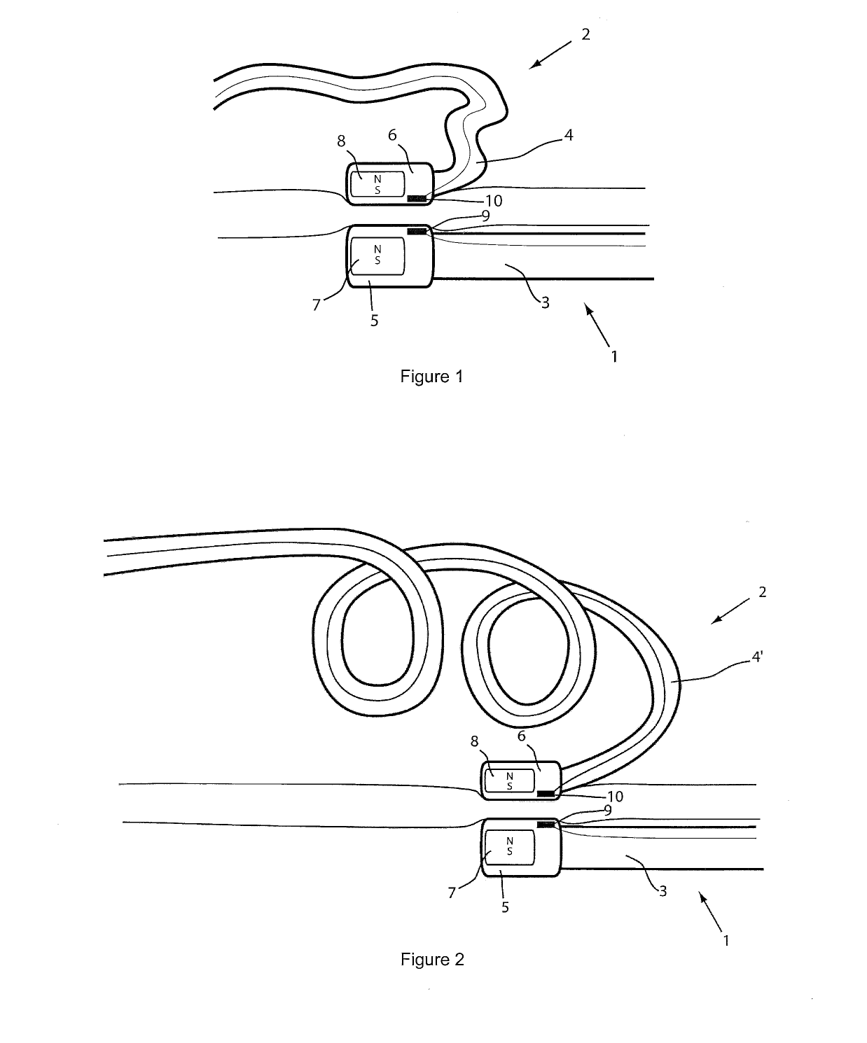

A first problem one has been confronted with when using the system described in WO 2008/010039 mentioned above is the “guidability” of the guided member. As indicated and described in this incorporated prior art, the idea then was to provide a system with two elongated members, used in particular for ablation, having at their distal end at least a magnet or a magnet arrangement for a magnetic coupling of said distal end when they are brought close together. Experiments with prototypes of the system described in the WO 2008/010039, show that the guiding member should be more rigid than the guided member to allow a proper functioning of the system. In fact, it was observed that the less rigid or the more flexible the guided member is, the better it follows the guiding member. A first aspect of the present invention therefore is the fact that the guided device has to possess a much greater flexibility (or much lesser rigidity) than the guiding member. This principle is illustrated in FIGS. 1 and 2 of the present application illustrating this first aspect. According to these figures, the device comprises a first catheter 1 (for example a guiding catheter) and a second catheter 2 (for example a guided catheter). As illustrated, each catheter 1, 2 comprises an elongated member 3, 4 (4′ in FIG. 2) at the distal end of which there is a head 5, 6, each head comprising a magnetic system 7, 8 (for example a magnet) and a sensor 9, 10 (for example a temperature and/or force and/or magnetic sensor). More specifically, since both members are firstly introduced individually in the human body, the guided member should possess a minimum rigidity to allow this introduction, but at the same time, when displaced by the guiding member according to the principle exposed in WO 2008/010039, it should have a reduced rigidity, preferably a minimal rigidity or highest possible flexibility to allow a proper guiding by the other member without interferences. This can be achieved, for example, by the use of temporary means or by a member that has different properties. In the FIGS. 1 and 2, this is illustrated by the different shapes of the elongated members 3 and 4, 4′, the member 3 belonging to the guiding element and the members 4, 4′ belonging to the guided member. Typically, the guided elongated members 4, 4′ may have different shapes as illustrated.

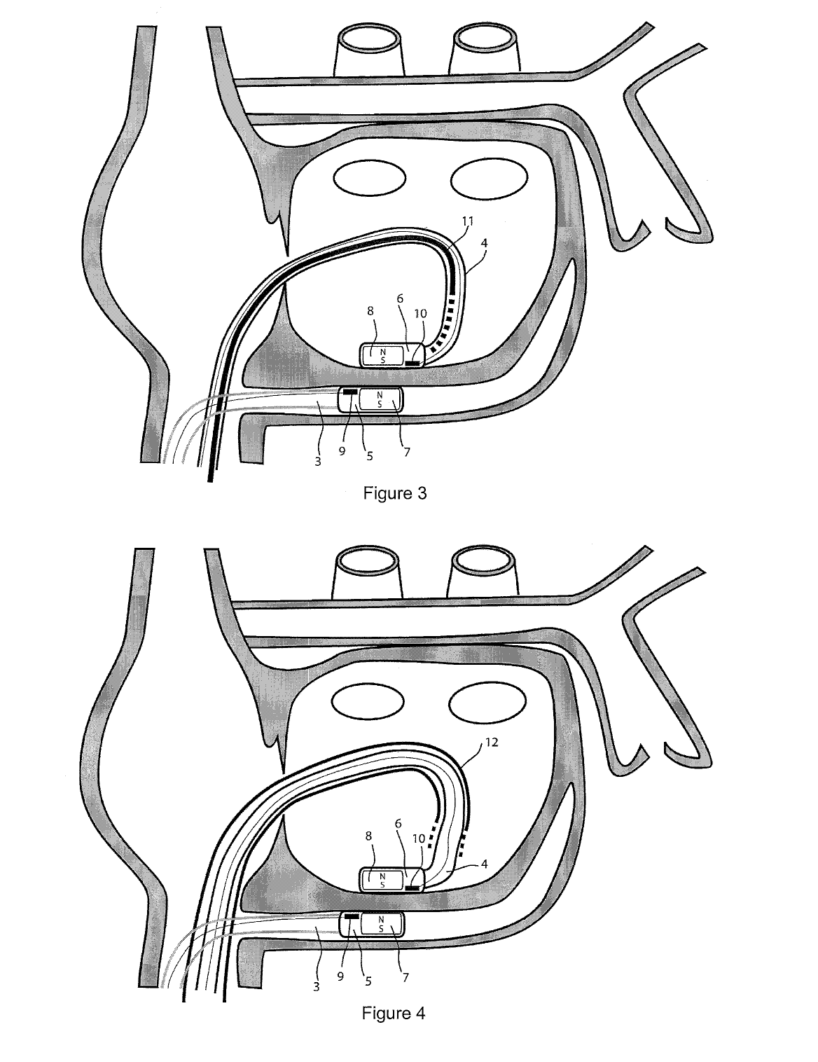

In FIG. 3, another embodiment of the present invention has been illustrated in position in the human body. According to this embodiment, a first member 3 (for example a guiding member), similar to the one illustrated in FIGS. 1 and 2 is introduced in a human body. This member 3 comprises at least at its distal end a magnetic system 7 (for example a magnet) and a sensor 9 (for example a temperature sensor). In this FIG. 3, there is also represented a second member 4 (the guided member in this representation) which comprises at its distal end a magnetic system 8 (for example a magnet) and a sensor 10 (for example a temperature sensor). To allow introduction of said second member 4 that is flexible according to the principle of the present invention into the human body, it is combined with a rigid inner member 11, or stiffening member, that is temporarily in place, i.e. mainly during the introduction of the member 4 in the human body to help the displacement of said member into places that may be tortuous. Once the member 4 is in place, the inner member 11 is removed at the proximal end of the member 4 and the member 4 can then be guided by member 3 as wished by the user, in the manner explained in WO 2008/010039. The inner member 11 could be also flexed at discretion of the operator and provided with means to these ends in order to better conform with the passages in which it is introduced and also to be easily guided in the human body.

In FIG. 4, a variant of FIG. 3 is illustrated. In this variant, the same elements are identified with the same references as in FIG. 3 and the description made above applies correspondingly to this variant. In this variant, instead of an inner stiffening member 11, one uses an outer stiffening sheath 12 for the same purpose. Once in place, the sheath 12 may be removed, for example at the proximal end of the member 4. The stiffening sheath 12 could also be flexed at discretion of the operator and provided with means to these ends for the same reasons indicated above for member 11. The systems illustrated in FIGS. 3 and 4 represent a further development of the concept explained in WO 2008/010039 according to which at least one of the members has the mean allowing to modulate the flexibility of its distal portion at discretion of the operator.

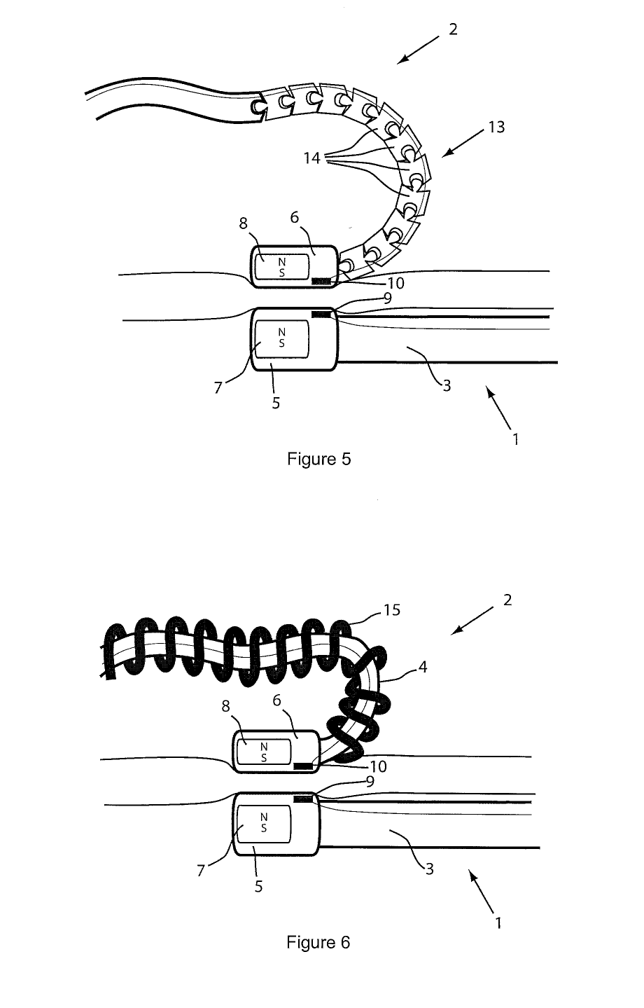

In FIG. 5, another variant is illustrated and similar elements are identified by the same references as in FIGS. 1 to 4 and the above description applies correspondingly. In this variant, the guided member 13 which comprises a tip 6 with magnetic means 8 and a sensor 10 comprises at least a part of its length made of individual elements 14 which are nested one into another or rigid elements separated by flexible joints which allow the member 13, or at least a part of it to be rigid in one direction and flexible in another direction as illustrated in FIG. 5. Such configuration will prevent the kinking of flexible part of the guided member while providing high flexibility.

In FIG. 6, a further variant is illustrated. In this variant again, similar elements are identified as in the preceding figures and the description applies correspondingly to these elements. In this variant, the outer “rigidifying” member 15 has the shape of a telephone cord and is wrapped around the member 4 to provide a sufficient rigidity upon insertion while providing flexibility during guiding movement. As described previously, it may be removed (for example proximally) once the member 4 is in place.

Of course, FIGS. 3 to 6 illustrate examples of means that can be used and/or combined for the mentioned purpose and other equivalent means alone or in combination may be envisaged in the frame of the present invention.

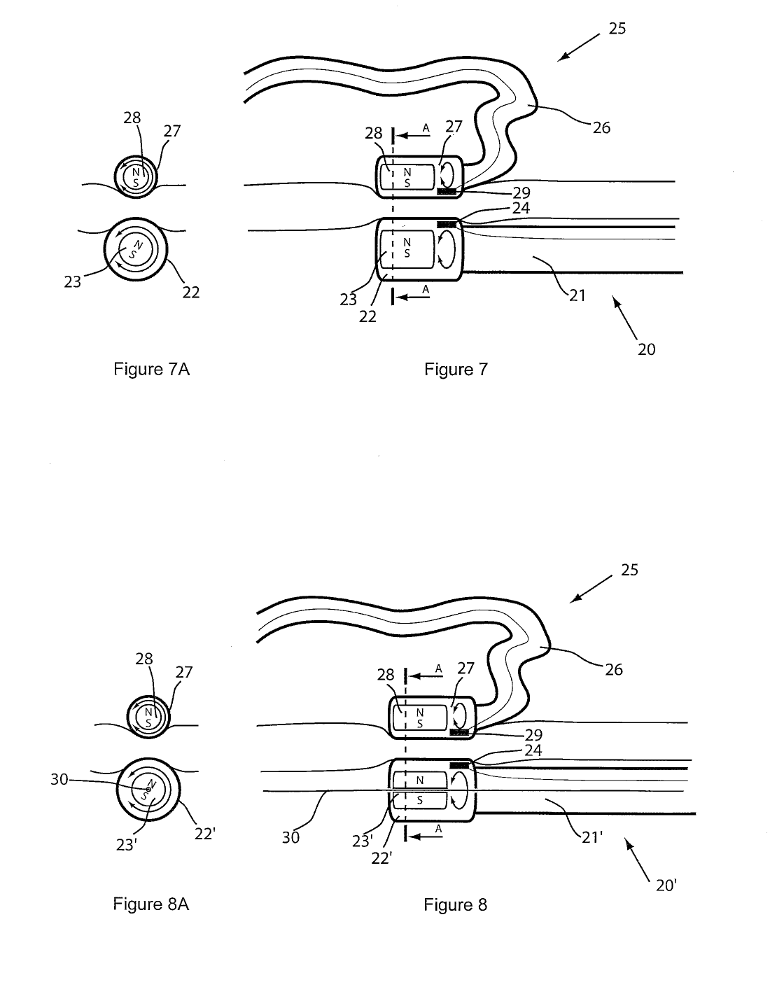

FIGS. 7, 7A and 8 and 8A illustrate other embodiments of the present invention. In FIGS. 7 and 7A, the first catheter 20 is a guiding catheter and comprises an elongated member 21, a distal tip 22 with a magnetic means 23 (for example a magnet) that is diametrically magnetized (meaning that the magnetic vector is directed along the magnetic means diameter, when the magnetic means have a cylindrical shape). It may also comprise a sensor 24, such as a temperature sensor. Similarly, the second catheter 25 is a guided catheter and comprises an elongated member 26, a distal tip 27 with a magnetic means 28, for example a magnet that is also diametrically magnetized, and may also comprise a sensor 29 (such as a temperature sensor). Specifically, in this embodiment and as illustrated in the FIGS. 7 and 7A by arrows, each magnetic means 23, 28 are mounted in a cage in which they are free to rotate. This allows them to take the best position for mutual attraction when they are aligned and this position is independent from the relative position of each catheter 20, 25. Accordingly, the catheters may also be rotated with respect to each other and this has no effect on the magnetic interaction, in particular, this does not reduce the magnetic force between the catheters. Of course, this principle may be applied in any of the preceding embodiments and variants described above. In addition, only one of the magnetic means may rotate or both as illustrated.

In FIGS. 8 and 8A, a variant of FIGS. 7, 7A is illustrated in which similar elements are identified by the same numerical references and the description made above applies correspondingly. In addition, in this variant, the catheter 20′ is combined with a guide wire 30 going through the member 21′ for helping to guide the movement of the catheter 20′. The guide wire 30 could pass through a lumen in the axis on which the magnetic means 23′ rotates and continue through the entire length or a part of the length of the member 21′ of the guiding member in a way known in the art as “over-the-wire” or “monorail”. Of course, it is possible to use such a guidewire 30 in other embodiments of the invention as described in the present application with corresponding adaptation to the design.

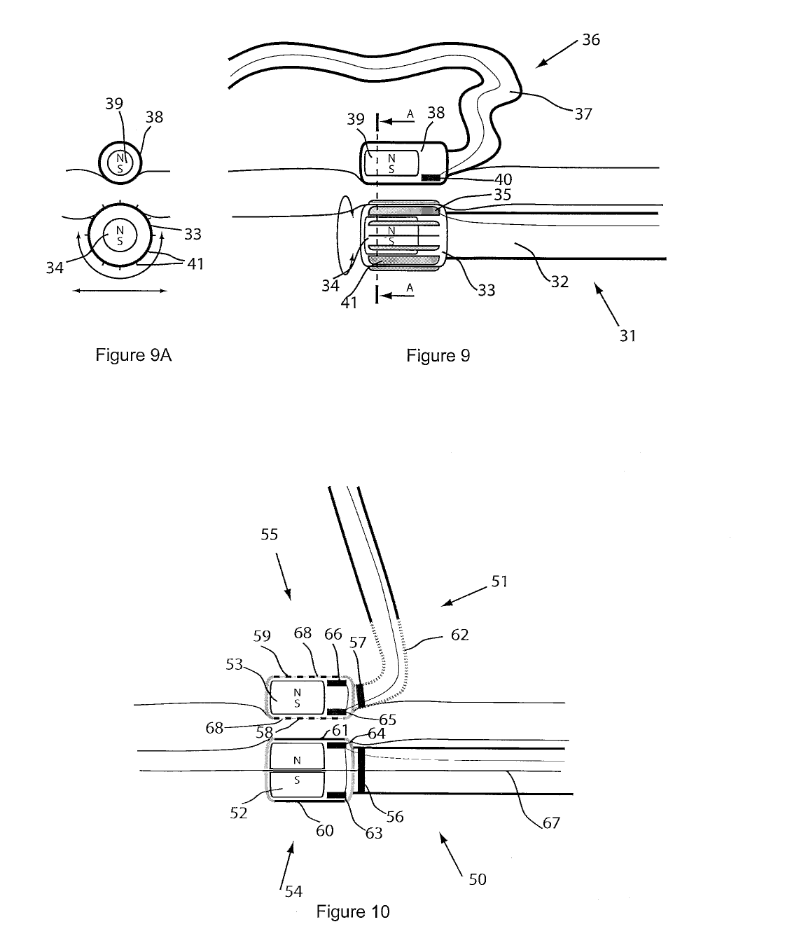

In FIGS. 9 and 9A, another embodiment of the present invention is disclosed. In this embodiment, there is, as in the preceding embodiments described above, a first catheter 31 which is used as a guiding member according to the principle of the present invention. As previously described, this catheter 31 comprises a member 32 and a tip 33 with magnetic means 34, such as a magnet, and may also include a sensor 35. The other catheter 36, for example the guided catheter, comprises a member 37 and a tip 38 with magnetic means 39, for example a magnet, and may also include a sensor 40. In addition, the distal tip 33 of the catheter 31 comprises a series of lateral fins 41 which are used as a grip means for helping the lateral displacement of the catheter 31. When axially rotating the catheter 31, the fins 41 grip into the body part that is present between the two catheters 31, 36 and is thus moved laterally as is illustrated in FIG. 9A. It could be very advantageous that only the distal tip 33 or its outer surface rotates while the catheter 31 remains without rotation. Of course, it is possible to use in this embodiment features of other embodiments, for example the rotating magnets of the embodiments of FIGS. 7, 7A, 8 and 8A or to combine said embodiment with other embodiments of the present invention described in the present application.

The magnetic catheters according to current invention provide an important advantage of self-orienting the tip surface in connection with North (N) or South (S) pole of the magnet towards the tissue surface. This feature is advantageously further used by the current invention. As an example, the surface of the ablating electrode may be limited only to the area of N or/and S pole of the tip therefore providing a greater electrical current density at the electrode-tissue interface. The remaining surface of the tip may be covered (insulated) by a non-conducting material. Other solutions are also possible: the tip could be constructed from two different materials (one conducting electricity and other dielectric) in a way that conducting part of the tip corresponds to the magnetic pole of the integrated magnet while the side parts of the tips are made from dielectric thus the alignment of the magnetic means would result in a proper positioning of the ablating electrodes.

In a same way the irrigation holes could be placed only on the area of N or S pole of the tip therefore providing selective delivery of the irrigation solution to the area of electrode-tissue interface.

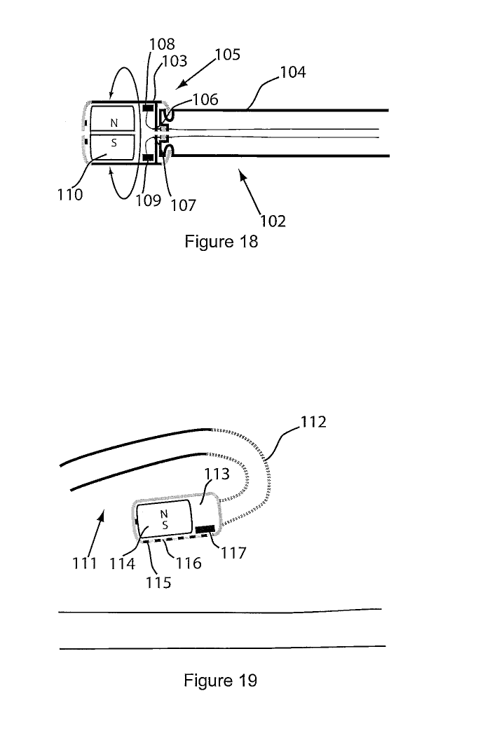

FIGS. 10 and 11 illustrate such embodiments of the present invention with like elements being referenced in a similar manner. Each catheter 50, 51 has a construction similar to the one previously described and contains magnetic means 52, 53 for example permanent magnets diametrically magnetized at its distal tip 54, 55 as described previously. In these embodiments, the magnetic means 52, 53 are not freely movable inside the distal tip 54, 55 of the catheters 50, 51 but are rather mechanically affixed within the distal tip 54, 55. In order to keep an easy self-coupling effect between both members, both distal tips 54, 55 are linked with the rest of the catheters 50, 51 by means of a free rotating joint or universal joint 56, 57 allowing free rotation of each distal tip 54, 55 over the axis of the catheter as further shown on the FIG. 18. Having magnetic means mechanically fixed with the rest of the distal tips 54, 55 offers only two possible configurations of magnetic coupling: S pole of the guided catheter with N pole of the guiding catheter or N pole of the guided catheter with S pole of the guiding catheter. This provides the opportunity to limit the functional surface of the catheter tip in direct contact with the tissue to essentially two areas situated right over the N or S magnet pole, for example areas 58, 59 of the guided catheter 51 and areas 60, 61 of the guiding catheter 50 as illustrated in FIG. 10, or at least to areas situated close or in vicinity of the poles. If, in this embodiment, the ablation catheter is preferably catheter 51 and the catheter 50 is the guiding catheter, then the areas 58, 59 may be RF electrodes used for ablation.

Preferably (see the embodiment of FIG. 10), a pre-curved flexible section 62 in the catheter 51 reduces the possible tips coupling combinations to a single one. This pre-curved section may be used in any embodiment of the present invention as described herein. As shown in FIG. 10, the flexible portion 62 of the distal part of the catheter 51 prevents coupling between the N pole of the tip 55 and the S pole of the tip 54 allowing only single coupling configuration possible (S pole of catheter 51 to the N pole of the catheter 50).

The embodiment of FIG. 10 allows limiting the functional surface of the tip 55 to only the area directly overlying the S pole of the tip magnetic means 53. This functional surface can accommodate an ablation electrode, irrigation holes (channels), temperature sensor(s), pressure sensors, other sensors and sources of ablating energy. Another possibility to allow a single magnetic orientation is to use sensors 63, 64, 65 and 66 such as force sensors or magnetic field sensors to detect which electrode 58, 59 or which part of the distal tip of the guiding catheter 50 is in contact with tissue. Also illustrated in FIG. 10 is a guidewire 67 that can be used as known in the art.

In the embodiment of FIG. 11, the guided catheter 51′ is slightly different than the one illustrated in FIG. 10 but works according to the same principles. In this embodiment, the guided catheter 51′ is frontally put in magnetic relation with the guiding catheter 50. The guided catheter 51′ which is preferably the ablation catheter comprises (as the catheter 51 as illustrated in FIG. 10) magnetic means 53′, sensor means 65′, 66′, and electrode 58′, 59′. On the electrode, there are in addition irrigation holes 68′. Similarly to the embodiment of FIG. 10, this embodiment may also comprise a free rotation joint or a universal joint allowing a rotation of the tip 55′ while still ensuring an electrical contact between the elongate member and the tip 55′.

A selective double or single magnetic coupling configuration provides the following advantages.

The first advantage relates to energy delivery efficiency. Limiting the ablation electrode surface to area directly overlying the N or the S magnetic pole ensures that the ablation electrode is only in contact with tissue and not with surrounding blood flow allowing to increase the electrical current density and the quantity of delivered energy directly to the tissue for the same electrical power. The remaining part of the tip is isolated (does not conduct electricity) and does not deliver the electric energy to surrounding environment (i.e. to blood). In a same way the use of other ablation energy sources such as cryo, microwave or ultrasound, laser or ionising radiation can easily be accommodated onto the functional surface overlying the N or the S magnet pole and deliver the energy selectively and directly to the tissue.

Likewise, the number of irrigation holes 68, 68′ normally present on the tip 55, 55′ of an ablation catheter 51, 51′ may be reduced and limited to the area of the tip connected to the S or N pole of the magnetic means 53, 53′. In FIGS. 10 and 11, the same area serving for delivery of RF energy to the tissue contains the irrigation holes 68, 68′. This second advantage results in directing the irrigation solution towards the tissue surface and lowering the amount of solution used for irrigation during the procedure.

The third advantage of placing the tip functionalities only to the surface connected to the magnet pole is that it provides new opportunities of measuring the ablation propagation and the biological status of the tissue or even the tissue thickness. Components 63, 64, 65′, 66′ could represent either sensors, or light sources or a combination of both. Temperature sensor, electrical impedance sensor, acoustic impedance sensor, or sensors measuring transmittance, reflectance or fluorescence of the tissue can be directed towards the tissue surface by N-S/S-N coupling of the tip magnets allowing to measure the heat and/or tissue damage propagation. The guiding catheter 50 in this case offers a novel possibility to measure thermal damage of the tissue through its entire thickness by measuring the surface temperature or transmittance or the acoustic impedance or the electrical impedance of the portion of tissue between the components 63, 64, 65′, 66′. In addition, the biological status of the tissue surface in contact with the guiding member can be determined using optical spectroscopy by studying the fluorescence and the radiance of the tissue surface. A change in the reflectivity of the tissue is correlated with thermal damage. Therefore, the distal part of the guiding member 50 can provide thermal damage information of the portion of tissue between the distal parts of the two catheters 50, 51, 51′.

For measuring tissue thickness, components 63, 64, 65′, 66′ may represent force sensors, pressure sensors, or magnetic field sensors. Knowing the physical characteristics of the magnetic means 52, 53, tissue thickness can be estimated from the force of coupling between the two catheters or from the magnetic field intensity measured by the sensors 63, 64, 65′, 66′.

According to the present invention, it is also possible to measure the tissue temperature by measuring the temperature of the magnetic means, for example permanent magnets. To realize this, the magnet should be able to move inside the tip in a way it can touch the surface of the tip, which is in contact with the tissue. The contact between the surface of the magnet and the inner surface of the tip represents a line. As the tip is made of thermal conductive material, the heat coming from the ablation process will easily be propagated in the tip and in consequence in the magnet. As air or vacuum is be present around the magnet in order to reduce the heat propagation coming from the blood, the temperature of the magnet is very similar to the one of the tissue. However, as the thermal properties of permanent magnets are poor, the magnets are preferably coated with a thermal conductive material such as gold for example. The sensor should be in close and good contact with the magnet but also isolated from the rest of the surrounding parts.

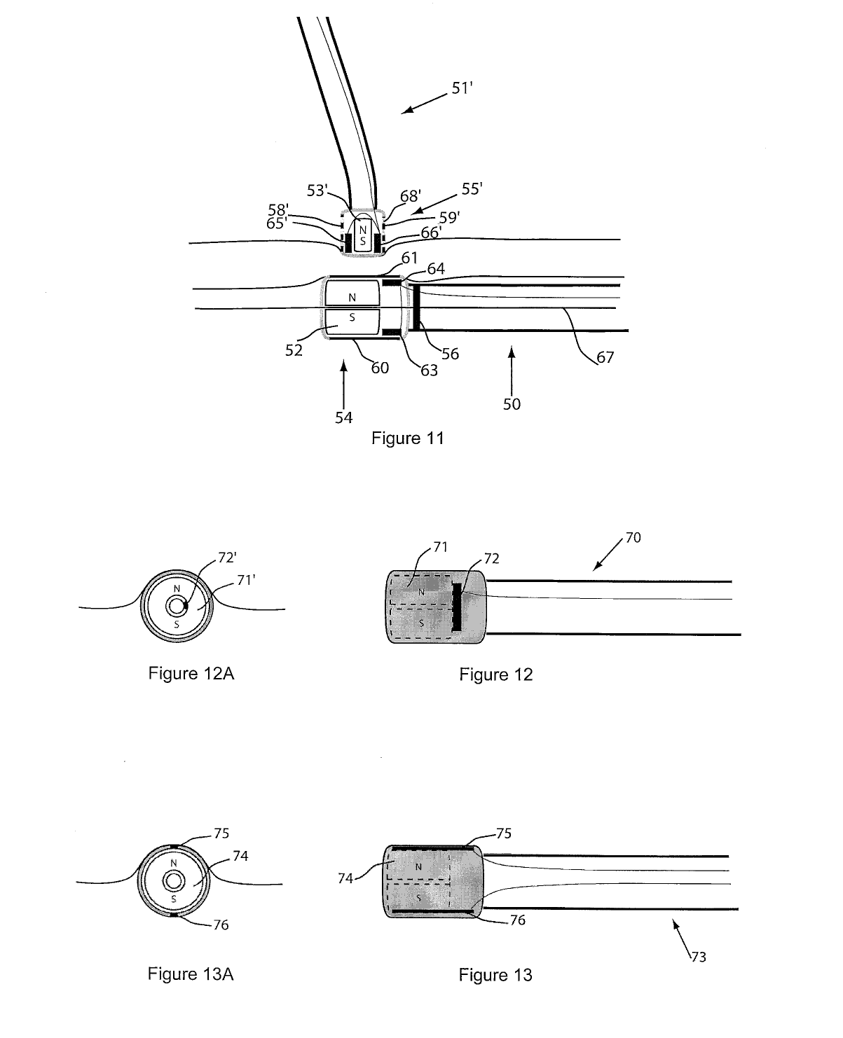

FIGS. 12 and 12A illustrate a variant of a catheter 70, preferably the guiding catheter that is not used in this configuration as the ablating catheter (this function being realized by the guided catheter not shown in this figure). In this variant, the catheter 70 comprises at its distal tip magnetic means 71, 71′ (such as a permanent magnet) and a temperature sensor 72, 72′. In FIG. 12, the temperature sensor 72 is placed at the back and/or in the front and/or inside a lumen of the magnetic means 71, and in FIG. 12A, the sensor 72′ is placed axially on the magnetic means 71′. Preferably, in the device according to the invention, the temperature sensor is at least placed in the catheter that is not the ablating catheter in order to give a precise measurement of the temperature at the ablation location. Preferably, the sensors are linked to the tip, i.e. if the tip is rotating they will also move with the tip.

In the variant of FIGS. 13 and 13A, the tip of the catheter 73 comprises magnetic means 71 (such as a permanent magnet) and two temperature sensors 75, 76 each placed close to the N and S poles of the magnetic means 74, this ensuring that a sensor is always close to the ablation site. As in FIG. 12, 12A, the catheter 73 is preferably not the ablating catheter, typically the guiding catheter.

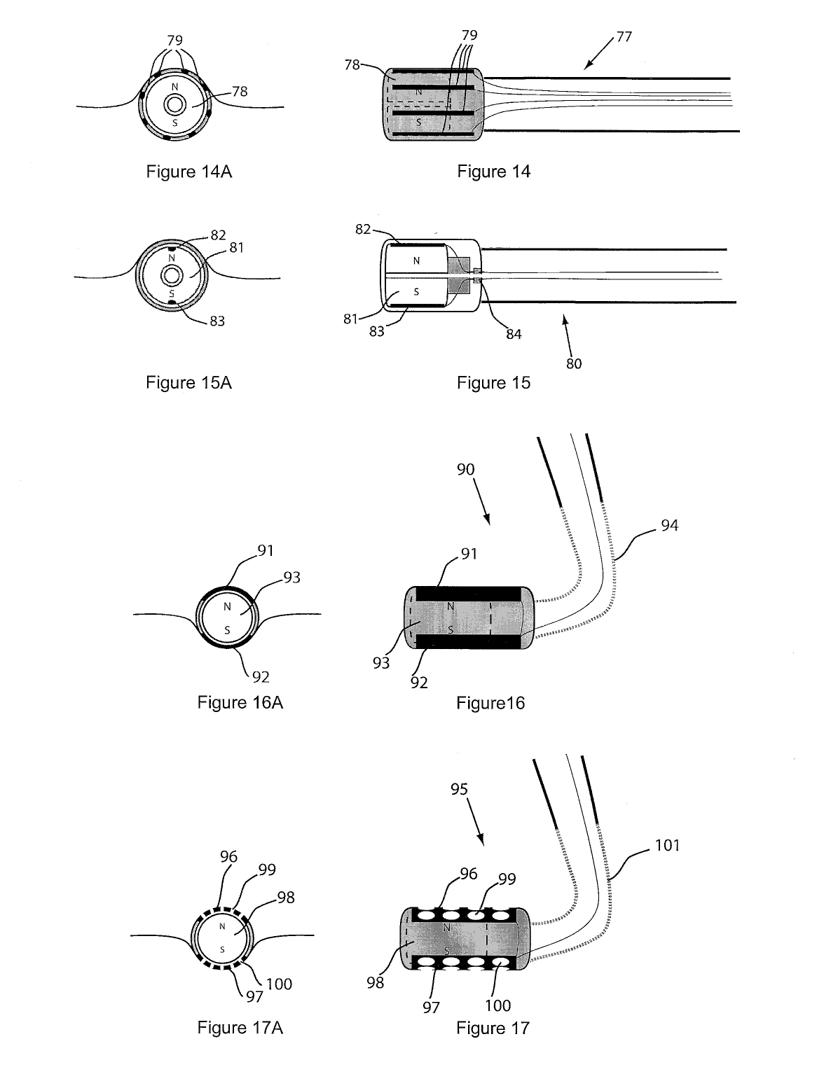

In the variant of FIGS. 14 and 14A, the catheter 77 comprises a tip with multiple temperature sensors 79 around the magnetic means 78 (for example a permanent magnet). This variant allows to make more precise measurements at different locations during ablation.

In the variant of FIGS. 15, 15A, the catheter 80 comprises two temperature sensors 82, 83 this time attached to the magnetic means 81 rather than to the tip as in FIG. 13. Since preferably the magnetic means rotate in the tip, the temperature sensors 82, 83 rotate as well with the magnetic means 81, To ensure an electrical connection, a rotating connector 84 may be used.

As mentioned above, the variants of FIGS. 12-15 (and 12A-15A) preferably illustrate the non-ablating catheter, for example the guiding catheter.

FIGS. 16 and 16A illustrate an embodiment of an ablation catheter according to the invention, In the example illustrated, the catheter 90 is an ablation catheter and its tip comprises ablation electrodes 91, 92 linked to the poles N or S of the magnetic means 93, over or close to the poles. Accordingly, in application with the principle of the present invention, an electrode 91 or 92 will be properly placed at the ablation site when the magnetic means of this catheter 90 and of the other catheter (not shown in FIGS. 16 and 16A) will cooperate together (see the above disclosure of the present invention). Preferably, the material of the tip that is around the electrodes 91 and 92 is dielectric in order to concentrate the electric field on the ablation site. Also illustrated in FIG. 16 is the preformed distal part 94 of the catheter. Preferably, in this embodiment, the tip of the catheter 90 may be free to rotate with the magnetic means 93 to allow the alignment of the poles N/S of the magnetic means 93.

In FIGS. 17 and 17A, a further variant is illustrated that is based on the variant of FIGS. 16, 16A. In this variant, the catheter 95 comprises a tip with ablation electrodes 96, 96 also linked to the poles N/S of the magnetic means 98 (for example a permanent magnet). In addition, the electrodes 96, 97 comprise irrigation holes 99, 100 for the passage of an irrigation fluid. The part of the tip not forming the electrodes is preferably formed of a dielectric material for the reasons indicated above. The catheter also includes in the representation a preformed distal part 101 for the reasons given above.

Another embodiment of the tip of a catheter is illustrated in FIG. 18. Preferably, but not limited thereto, the catheter 102 illustrated is not the ablating catheter, and for example the guiding catheter. More specifically, it illustrates the free rotation link 105 between the tip 103 and the body 104 of the catheter 102. Preferably, this link 105 comprises rotating electrical connectors 106, 107 to ensure signal transmission during rotation. Also illustrated are sensors 108, 109 for example temperature sensors, and magnetic means 110, for example a permanent magnet. In this embodiment, as one will understand, the tip 103 is able to rotate with respect to the body 104 of the catheter 102 so the magnetic means 110 may be fixed in the tip 103. Also, one will understand that this embodiment may be used in other various embodiments of the invention as disclosed herein.

FIG. 19 illustrates an embodiment with the preshaped distal part 112 of the catheter 111 (preferably a guided catheter) when the catheter is under no constraint. The curve is made in such a way which allows only one half of the tip or magnetic pole of the magnetic means 114 to be in contact with tissue, meaning coupled with a magnetic pole of the guiding catheter (not represented in this figure). As a typical example, this catheter 111 may be used in combination with the catheter 102 illustrated in FIG. 18, the catheter 111 being the guided (ablation) catheter and the catheter 102 being the guiding catheter. Of course, this is only an exemplary combination and other are possible within the frame of the present invention.

The limitation of movement of the catheter of FIG. 19 due to the preshaping with a non-rotating tip 113 may be used as an advantage because the ablation electrode 115 and the irrigation holes 116 as well as sensors 117 can be present on only one of the magnetic poles. Using the catheter 102 of FIG. 18, one ensures that the best magnetic coupling is realized since the tip 103 may rotate and the poles N/S of the magnetic means 110 and 114 may align themselves properly.

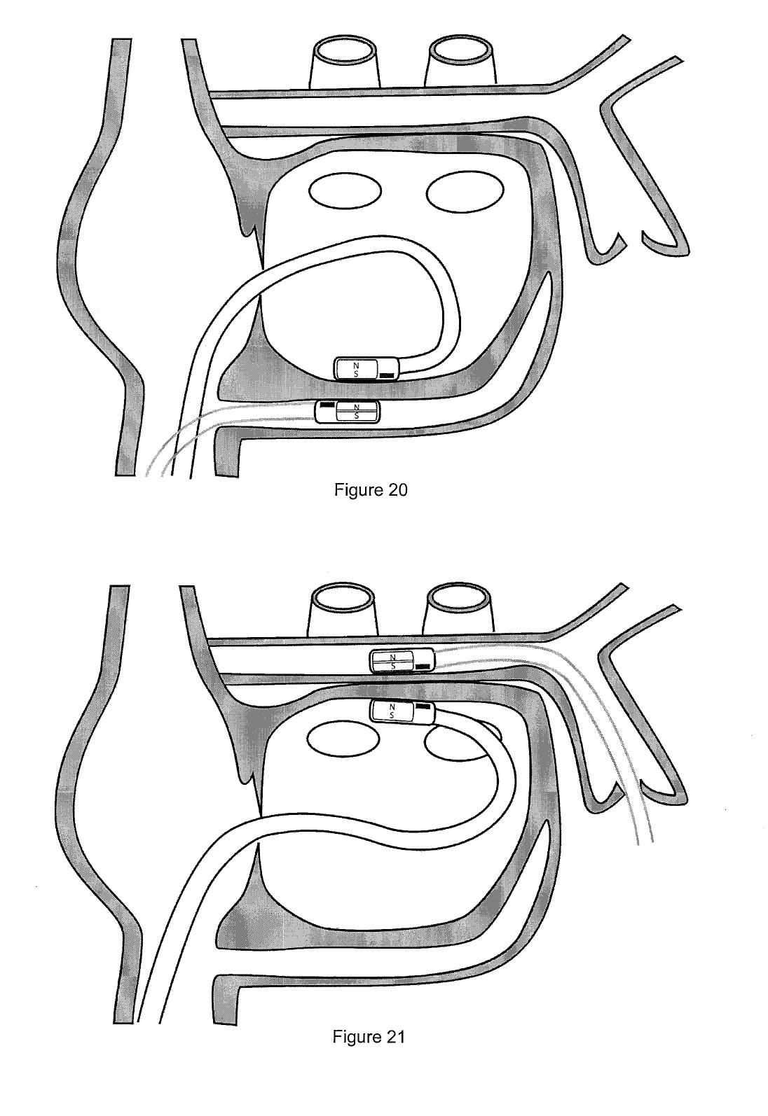

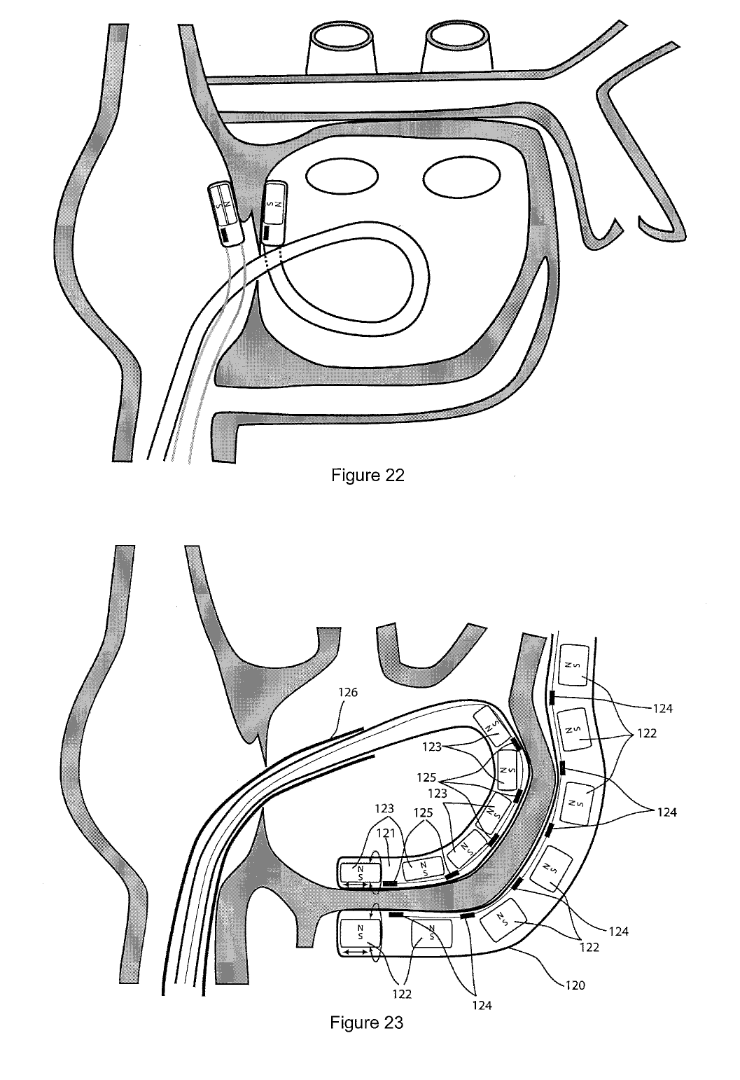

FIGS. 20 to 22 illustrate different medical ablation procedures that may be undertaken with the device of the invention, typically in FIG. 20 a first example (coronary sinus line) of the catheters configuration during the ablation procedure, in FIG. 21 a second example (pulmonary artery) of the catheters configuration during the ablation procedure and in FIG. 22 a third example (septum line) of the catheters configuration during the ablation procedure.

As a skilled man will understand, any embodiments of catheters as described herein may be used in these application examples and the illustration should not be construed in a limiting manner.

Another embodiment of the invention is illustrated in FIG. 23. Each catheter 120, 121 comprises a plurality of magnetic means 122,123, for example permanent magnets, which are meant to cooperate magnetically with each other (for example by a coupling) trough human tissue. To the difference with the known previous art, the magnetic means are preferably permanent magnets of cylindrical shape diametrically magnetized (meaning that the magnetic vector is directed along their diameter). In addition, the magnets 122, 123 are freely movable inside the member either in axial rotation and/or axially. This is illustrated by the arrows on the distal magnets 122, 123 of members 120, 121 in FIG. 23. Of course, this feature applies preferably to all the magnets of each catheter 120, 121. Such free rotating magnets allow easy self-coupling between both members in a way that an operator does not need to rotate the catheters 120, 121 to reach the coupling. This also allows to maintain the coupling force even if one of the catheters or both are rotated relatively one to another which is often the case in tortuous cardiac anatomies.

In the embodiment of FIG. 23, the device may in addition comprise sensors 124, 125 preferably temperature sensors, which are used to measure the temperature during ablation. Such sensors are at least placed on one side of the human tissue, most preferably on the other side with respect to the ablation element or on both sides. As described in WO 2008/010039, ablation may be carried out with RF means. More advantageously in the current application, cryo energy could be applied through the ablation device according to the invention in place of RF energy. Measuring of the temperature on the opposite side and adjusting the quantity of the cryo energy delivered accordingly will allow formation of the cryo lesion through the entire depth of the wall being treated. Also some sensors 124, 125 may be used as pressure sensors to give an indication of the pressure applied to the tissue between the two members. Also, a combination of sensors (pressure, temperature) may be used.

Due to the presence of a plurality of successive magnetic means (i.e. magnets) 122, 123, the system places in a stable manner the two members which undergo a magnetic coupling between them. The geometry allows such coupling over a certain distance which in turn allows an ablation over said distance as well when using several ablation elements. To allow such ablation, the sensors 124, 125 are preferably temperature sensors to properly monitor the temperature at the ablation site(s). Of course, to this effect, it is necessary to use several ablation means which are distributed along one of the members and not a single ablation means that would for example be placed at the distal tip of one of the members.

To help the guiding and placement of catheter 121 before ablation, one preferably uses an external guiding relatively rigid sheath 126 that can be removed once the catheters are properly positioned to then carry out the ablation step. Of course other equivalent means may be used to help bringing the catheter(s) in position (guidewires etc).

As will be understood, the embodiments of the invention described in relation to the previous figures may also be used in the embodiment illustrated in FIG. 23 with corresponding adaptation if needed.

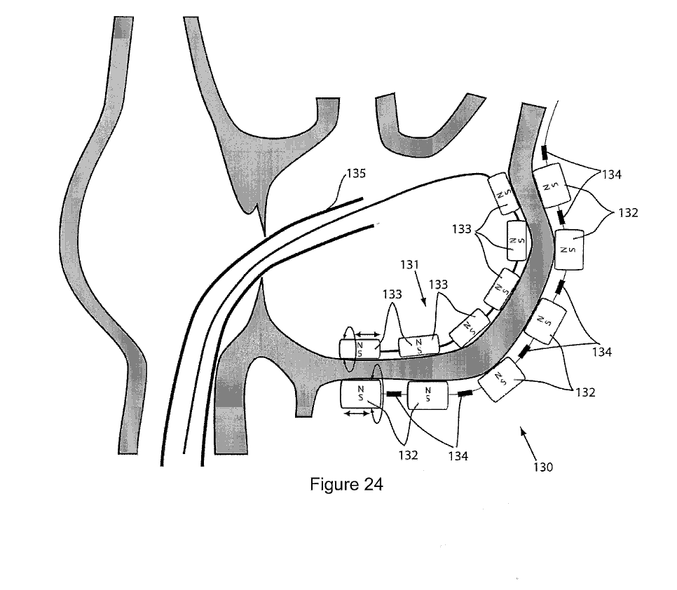

FIG. 24 illustrates a further embodiment in which the catheters have the shape of a “chain” or a “necklace” 130, 131 of several successive magnetic devices 132, 133 (for example magnets). One of the members in addition comprises sensors 134, i.e. temperature sensors to monitor the temperature at the ablation site. Preferably, the sensors 134 are placed on the other side of the ablation site with respect to the ablation means. In this embodiment, the magnetic means 132, 133 are also preferably free to move axially or to rotate as in the previous embodiment, this feature being schematically illustrated for the magnets placed at the distal end of each member 10, 12. Of course, this feature is preferably present for all magnetic means.

This could also be advantageously used in order to actively displace (for example rotate) a magnetic means or magnetic means assembly inside a cage or to deliver a back-and-forth movement to a magnetic means or magnetic means assembly at the tip of the catheters with the purpose to decrease the magnetic interaction between the catheters if necessary. Preferably, in this embodiment an additional sheath 135 is used to bring the catheter 131 in position and then the sheath is removed for example proximally. A similar sheath may of course be used to bring the other catheter 130 in position, said other sheath being removed once the catheter is in position.

As will be understood, the embodiments of the invention described in relation to the previous figures may also be used in the embodiment illustrated in FIG. 24 with corresponding adaptation if needed.

Thanks to this medical device, and method, a complete control over the ablating tip is achieved and allows the generation of precise continuous lines of ablation in the region to be treated, while minimizing the risk of thermal injury to the regions to be treated thanks to a precise measure of the temperature of the tissues in the vicinity of the region to be treated.

While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

For example, the magnetic means, for example the magnets, may not only be allowed to rotate as illustrated in the figures but they may also have a longitudinal play to further facilitate and improve the coupling of the members. This could also be advantageously used in order to actively displace (for example rotate) a magnet or magnets assembly inside the cage or to deliver a back-and-forth movement to a magnet or magnets assembly at the tip of the members with the purpose to decrease the magnetic interaction between the members if necessary.

Also, any combination of the different embodiments and variants described above may be envisaged and chosen, according with the circumstances.

Permanent magnets may be used as magnetic means or other equivalent means allowing a coupling of the members and a guiding in accordance with the teaching of the present invention.

In addition, irrigation means may be used with the present invention, as described in WO2008/010039 for cooling and cleaning purposes, as has been described above. To this effect, the concerned catheter will comprise irrigation holes and at least a lumen. In this event, preferably, there will be at least a temperature sensor on the other catheter (not the one used for irrigation) to measure the temperature at the ablation site. Preferably, the irrigation catheter is the guided catheter if said catheter is the ablation catheter.

Of course, any other feature disclosed in WO 2008/010039 incorporated by reference in its entirety in the present application may be used in the device according to the present invention.

Many further variants and embodiments may be envisaged for the present invention as described herein. In addition to the combination of embodiments and variants, it is possible to implement other aspects. For example, it is possible to adjust the magnetic coupling either by using variable magnetic means, or, if permanent magnet are used, to adjust the coupling by deflecting the magnetic field. This can be done for example with a ferromagnetic cylinder that is moved over the magnets. Alternatively, one may use a movable magnetic bar to induce the same effect. Another variant is to use coils and ferromagnetic elements in close proximity of the magnetic means in order to create additional magnetic fields reducing the total mutual attraction between the catheters.

As mentioned above, sensors may be used to measure the magnetic/force coupling between the members. In addition to security purposes, this measurement may also be used to modulate the coupling forces between the members in order to optimize the displacement of coupled members.

1. A medical device for performing tissue ablation in a body, comprising a guiding member to be introduced in a first region of the body and a guided member to be introduced in a second region of the body, wherein at least one of said members comprises a sensor, such as a temperature sensor, wherein at least one of said members comprises an ablation means, wherein each said member comprises at least at its distal tip at least one magnetic means for allowing magnetic coupling between said members at the ablation site, and wherein the guided member is less rigid than the guiding member.

2. The medical device as defined in claim 1, wherein the ablation means are placed on the guided member and the sensor is a temperature sensor and is placed on the guiding member.

3. The medical device as defined in claim 1, wherein ablation means are placed on each member and wherein each member comprises a temperature sensor.

4. The medical device as defined in claim 1, wherein said magnetic means is movable relatively to said member.

5. The medical device as defined in claim 4, wherein said magnetic means may have a rotational and/or longitudinal relative movement.

6. The medical device as defined in claim 1, wherein said ablation means is situated in the vicinity of a pole of said magnetic means.

7. The medical device as defined in claim 1, wherein said sensor is situated in the vicinity of a pole of said magnetic means.

8. The medical device as defined in claim 1, wherein it comprises several temperature sensors.

9. The medical device as defined in claim 1, wherein said ablation means comprises irrigation holes.

10. The medical device according to claim 9, wherein the irrigation holes are situated in the vicinity of a pole of said magnetic means.

11. The medical device as defined in claim 1, wherein said guided member comprises a preshaped distal portion.

12. The medical device as defined in one of the claim 1, wherein it comprises additional means for rigidifying at least said guided member temporarily while it is being introduced in the body, said additional means being removable once the member is in position.
